(19) United States
(12) Patent Application Publication
(10) Pub. No.: US 2009/0256085 A1
(43) Pub. Date: Oct. 15, 2009

(54) Ultra-violet sponge holder

(76) Inventor: Devaraj Thiruppathi, Columbus, GA (US)

Correspondence Address: DEVARAJ THIRUPPATHI, 7219 Stone Creek Lane, Columbus, GA, 31909, US

(21) Appl. No.: 12/082,451

(22) Filed: Apr. 11, 2008

(51) Int. Cl. A61L 2/10

(52) U.S. Cl. 250/492.1; 422/24

(57) ABSTRACT

A ultra-violet sponge holder that emits sufficient radiation to eradicate germs, bacteria, viruses and other pathogens and microorganisms on a kitchen sponge with a ultra-violet light located on a holder top which is powered by a rechargeable battery with a on/off/auto switch with a safety mechanism whereby only turning the ultra-violet light on when the holder top is closed, also the holder top contains a holder top window whereby allowing the ultra-violet sponge holder to be mobile and a holder bottom contains a sponge holder and a drainage retention area where liquid from sponge is collected.

### FIELD OF THE INVENTION

The invention relates generally to a ultra-violet emitting sterilization device used to disinfect kitchen dishwashing sponges.

### BACKGROUND OF THE INVENTION

Kitchen sponges are the number 1 source of germs in the house and are responsible for as many as 80 million Americans contacting food-borne illnesses each year and of those 9,000 die. Ultra-violet radiation is well known to killing germs and viruses in the radiation range of 300-200 nanometers short wavelengths. Ultra-violet lamps have been used to eradicate germs, purifying air, water and surfaces. The intensity of the radiation and duration of exposure determines the potency of the ultra-violet radiation. The shorter the wavelength the stronger and quicker the kill factor. Ultra-violet light with wavelength shorter than 300 nanometer is extremely effective in killing microorganisms. The most effective sterilizing range of UV is within the C bandwidth of 253.7 nm. This range is called germicidal UV bandwidth or UVC. At present the kitchen sponges are disinfected by way of the microwave oven, which is hazardous to the sponge and by way of submerging the sponge in a bleach solution, which is time consuming. Also ultra-violet radiation is used in medical, environmental and recreational environments, such as hospitals, dental practices and tanning beds, granted a different intensity of ultra-violet radiation and duration is used in each category to obtain the desired effect. There are many hand held devices which emits ultra-violet radiation. U.S. Pat. No. 5,920,075 issued to Whitehead (Jul. 6, 1999) discloses a device with sufficient intensity to kill germs and bacteria in a large room with a combination unlocking mechanism. U.S. Pat. No. 4,952,369 issued to Beliloa (Aug. 28, 1990) describes a pocket size portable ultra-violet flash light device that kills germs by shining and passing the ultra-violet radiation over the surface. The flash light includes a housing and a ultra-violet light source an on/off switch and a gravity switch that limits the activation of the ultra-violet light source to the position wherein the light source is facing downward. U.S. Pat. No. 4,471,226 issued to Wisnosky, Mclaughlin and McGinn III (Sep. 11, 1984) describes a portable hand held device with a safety percussion which is force to activate only on flat surface.

There is a need for a ultra-violet sponge holder used to disinfect kitchen sponges having an ultra-violet light source with sufficient intensity to kill germs, bacteria, viruses and other pathogens and microorganisms.

### SUMMARY OF THE INVENTION

This invention relates to a kitchen sponge sterilization holder that emits ultra-violet radiation in a range sufficient to destroy germs, bacteria, viruses, pathogens and microorganisms. The ultra-violet sponge holder includes a housing containing a ultra-violet light source a power source, a sponge holder and a drainage retention area to hold drainage from a sponge.

The ultra-violet sponge holder is in the form of a container having a holder top containing the housing for the ultra-violet light source and a holder bottom containing the power source, the sponge holder and the drainage retention area. The housing containing the ultra-violet light source is located on the holder top and is held by a ultra-violet socket allowing for sterilization of sponge without damaging the ultra-violet light source from the drainage from the kitchen sponge. The drainage from the kitchen sponge is retained in the drainage retention area, which is also sterilized by the ultra-violet light and can be drained out at a later time. The ultra-violet light source is automatically activated for a short period of time when a on/off/auto switch is moved to the “auto” position causing the ultra-violet light to activate when the holder top is opened and then closed allowing for ample time for the sterilization of the kitchen sponge placed on the sponge holder. The ultra-violet sponge holder contains a reflective top coating and a reflective bottom coating on the inside of container allowing for a larger sterilization area of sponge by reflecting the ultra-violet light. The ultra-violet light source can also be turned on by moving the on/off/auto switch to the “on” position for a longer exposure of the sponge to the ultra-violet light and then turned off by either moving the on/off/auto switch to the “auto” position, which has a predetermined timing of turning on and off the ultra-violet light when the holder top is opened and closed, or moving the switch to the “off” position to turn the ultra-violet light off. The ultra-violet light source is powered by a battery source, which is a rechargeable battery located on the holder bottom and also having a rechargeable site to charge the battery by plugging into an outlet if one is available. The ultra-violet sponge holder can also have a removable sponge holder, which can be removed from the ultra-violet sponge holder containing a sponge holder drainage openings, which allows for drainage of liquids from sponge, in which not only sponges can be placed within the ultra-violet sponge holder to be sterilized but other items as well such as utensils and wash rags. The ultra-violet sponge holder can also have a holder bottom drainage openings to allow the liquid which is drained from the sponge to the drainage retention area to be drained away for the ultra-violet sponge holder. Multiple ultra-violet lights can be added to increase the sterility of the sponge such as adding a bottom ultra-violet light to the holder bottom allowing the sponge to be radiated by ultra-violet light from both side.

The ultra-violet sponge holder can also be utilized in the form of a hand held sterilization device to clean kitchen countertops and the like. The holder top contains a holder top window, which can be opened and closed to expose the ultra-violet light source to the external environment to sterilize other areas of the kitchen or house. Unplugging the power cord from the ultra-violet sponge holder and holding the ergonomic base of the holder bottom allows the ultra-violet sponge holder to be mobile and moving the on/off/auto switch to the “no” position activates the ultra-violet light to begin sterilization of other surfaces exterior to the ultra-violet sponge holder and moving the on/of/auto switch to the “off” position turns the ultra-violet light off.

Therefore, in accordance with present invention the ultra-violet sponge holder sterilizes kitchen sponges containing the sponge holder and liquid retaining area and containing an open and closing window on holder top to sterilize other surfaces exterior to the ultra-violet sponge holder. These and other objects, features and advantages of the present invention will become apparent from the following detailed description. It should be understood, however, that the detailed description and the specific examples, while indicating preferred embodiments of the invention, are given by way of illustrations only, since various changes and modifications within the spirit and scope of the invention will become apparent to those skilled in the art.

### BRIEF DESCRIPTION OF THE DRAWINGS

The present invention will become more fully understood from the detailed description given herein below and the accompanying drawings, which are given by way of illustration only and are not limitative of the present invention, wherein:

FIG. 1 is a plan view of the ultra-violet sponge holder with lid open exposing sponge;

FIG. 2 is a plan view of the ultra-violet sponge holder with lid open without sponge;

FIG. 3 is a top view of ultra-violet sponge holder with lid open without sponge;

FIG. 4 is a plan view of ultra-violet sponge holder with lid closed;

FIG. 5 is a cross sectional view of ultra-violet sponge holder with sponge;

FIG. 6 is a plan view of bottom of ultra-violet sponge holder;

FIG. 7 is a plan view of ultra-violet sponge holder with lid closed with a holder top window closed;

FIG. 8 is a plan view of ultra-violet sponge holder with lid closed and holder top window open;

FIG. 9 is a plan view of ultra-violet sponge holder with a holder bottom drainage;

FIG. 10 is a cross sectional view of ultra-violet sponge holder with two ultra-violet lights;

FIG. 11 is a plan view of ultra-violet sponge holder with a removable sponge holder;

FIG. 12 is an exploded view of ultra-violet sponge holder with a removable sponge holder;

FIG. 13 is a cross sectional view of ultra-violet sponge holder with a removable sponge holder with sponge:

### DETAILED DESCRIPTION OF THE PREFERRED EMBODIMENT

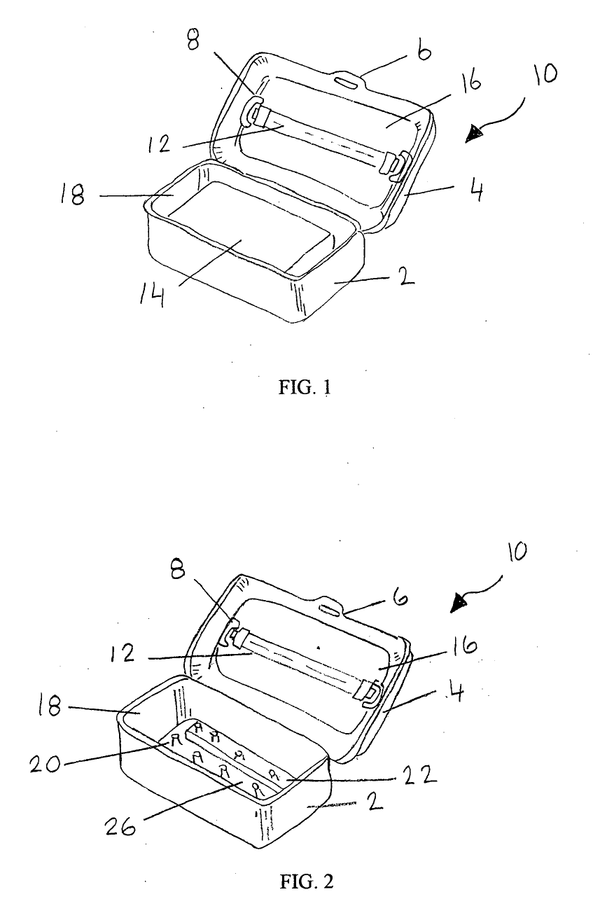

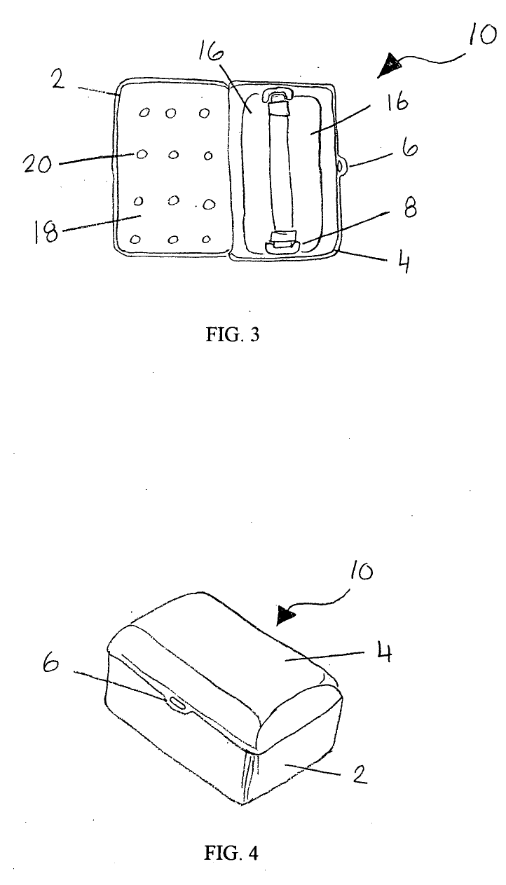

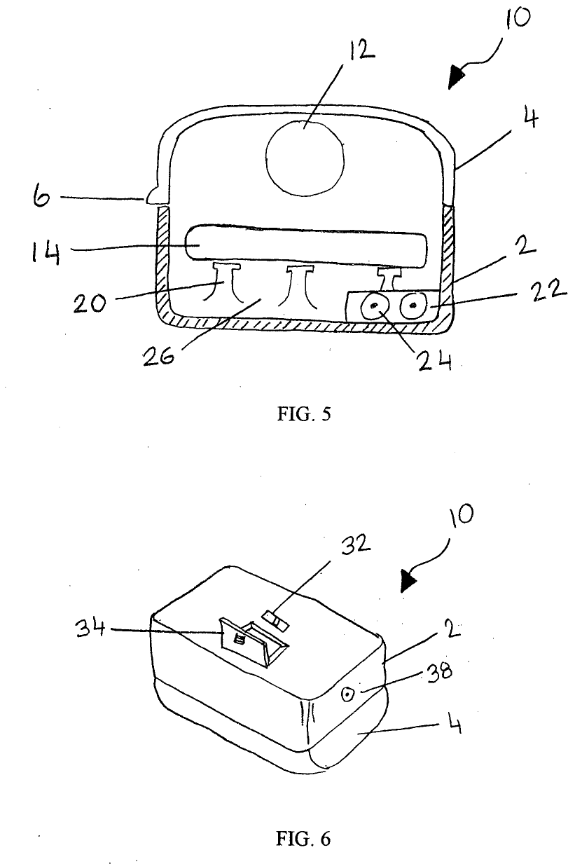

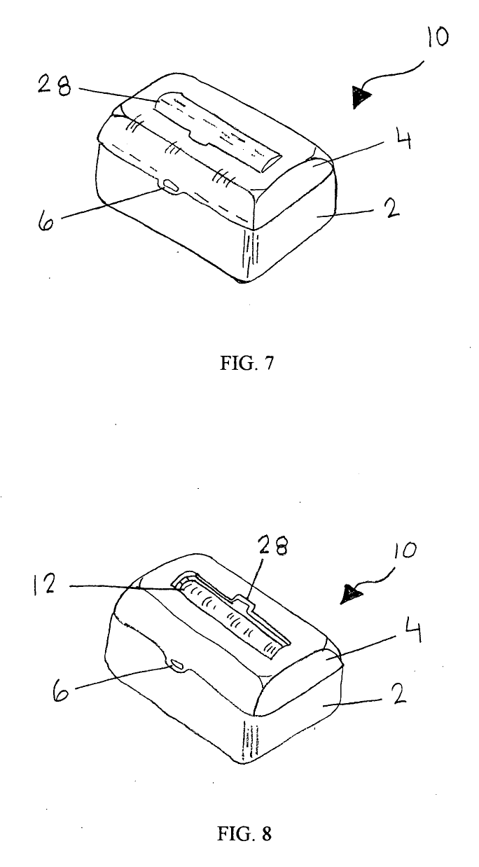

Referring now to the drawings wherein the depictions are for purposes of illustrating a preferred embodiment of the present invention only and not for the purpose of limiting the same. FIG. 1 is a plan view of a ultra-violet sponge holder 10 of present invention. FIG. 1 depicts the ultra-violet sponge holder 10 with a holder top 4 open exposing a sponge 14. The holder top 4 contains a ultra-violet light 12 which is being held in a ultra-violet socket 8 which through a electrical process aid in the illumination of ultra-violet light 12. There is a reflective top coating 16 and a reflective bottom coating 18 which helps radiate the sponge 14 located in a holder bottom 2 when the ultra-violet light 12 is illuminated. FIG. 2 is a plane view of ultra-violet sponge holder 10 without sponge 14, exposing a sponge holder 20 on which sponge 14 sits. The holder bottom 2 contains a drainage retention area 26 where the liquid from the sponge 14 sitting on sponge holder 20 drains into. FIG. 2 also depicts a battery container 22, which holds the power source for the ultra-violet light 12. FIG. 3 is a top view of ultra-violet sponge holder 10 depicting the holder top 4 and holder bottom 2. FIG. 4 is a plan view of ultra-violet sponge holder 10 with the holder top 4 closed onto holder bottom 2 with a holder tab 6 used to aid in the opening and closing holder top 4. FIG. 5 is a cross sectional view of ultra-violet sponge holder 10 with the holder top 4 closed on top of holder bottom 2. Holder top 4 contains ultra-violet light 12 and holder tab 6. Holder bottom 2 contains sponge 14 sitting on sponge holder 20 depicting drainage retention area 26 and battery container 22 containing a battery 24, which is the power source for the ultra-violet light 12 and is rechargeable. FIG. 6 is a plain view of holder bottom 2 of ultra-violet sponge holder 10. The holder bottom 2 consists of a battery container door 34 where the battery 24 is inserted into the battery container 22. The battery 24 can be a rechargeable battery and by rechargeable means be charged within ultra-violet sponge holder 10 by a rechargeable site 38 where an electrical cord plugs into. The holder bottom 2 consists of a on/off/auto switch 32 which controls the ultra-violet light 12, moving the on/off/auto switch 32 to the “on” position turns on the ultra-violet light 12 of ultra-violet sponge holder 10 and stays on until the on/off/auto switch 32 is moved to the “off” position, whereby deactivating the ultra-violet light 12 of ultra-violet sponge holder 10. The “auto” position only activates the ultra-violet light 12 of ultra-violet sponge holder 10 for a short period of time, when the holder top 4 is opened and then closed to sterilize the sponge 14 within ultra-violet sponge holder 10 as in FIG. 4. As a safety mechanism the ultra-violet light 12 can only activated when the holder top 4 is closed no matter at what position the on/off/auto switch 32 is placed.

FIG. 7 is another embodiment of ultra-violet sponge holder 10 in which the holder top 4 contains a holder top window 28, in a closed position. The holder top window 28 allows the ultra-violet light 12 to be exposed to the exterior environment whereby allowing the ultra-violet sponge holder 10 to be mobile and sterilize countertop surfaces, cutting boards and other exterior surfaces. FIG. 8 is a plan view of ultra-violet sponge holder 10 with the holder top window 28, in the open position, exposing the ultra-violet light 12 to the exterior environment and activating the ultra-violet light 12 by turning the on/off/auto switch 32 to the “on” position which turns the ultra-violet light 12 on and keeps it turned on until the on/off/auto switch 32 is moved to the “off” position.

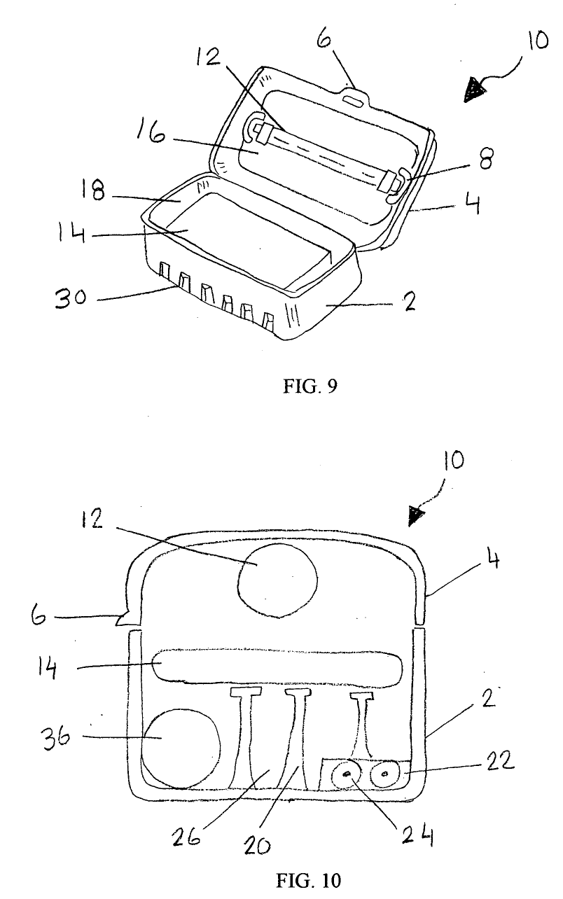

FIG. 9 is a plane view of still another embodiment of ultra-violet sponge holder 10 with a holder bottom drainage 30. The holder bottom drainage 30 contains opening located on bottom side of holder bottom 2 allowing the drained liquid from sponge being held in the drainage retention area 26 to flow out of ultra-violet sponge holder 10. FIG. 10 is a cross sectional view of yet another embodiment of ultra-violet sponge holder 10 with a bottom ultra-violet light 36. More then one ultra-violet light maybe required to sterilize sponge 14 bottom and top without turning sponge 14 over.

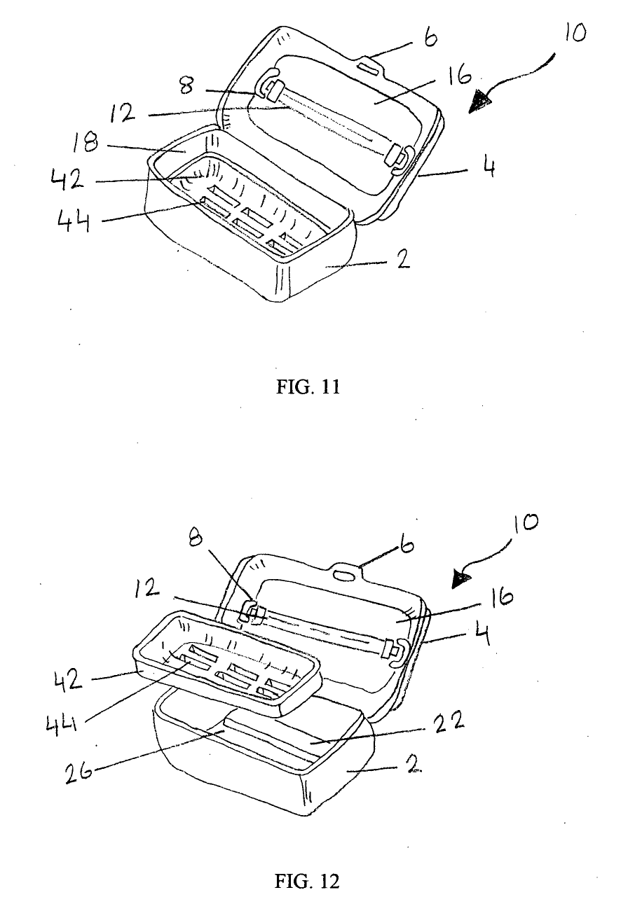

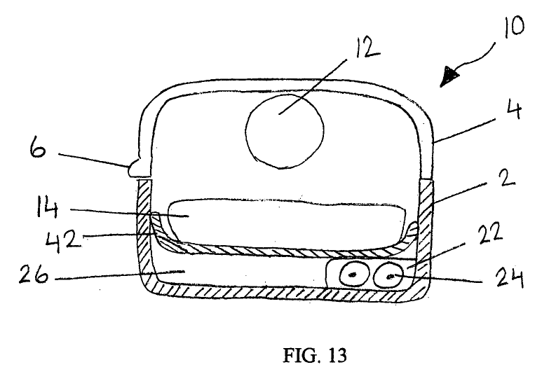

FIG. 11 is plan view of yet another embodiment of ultra-violet sponge holder 10 with a removable sponge holder 42. The removable sponge holder 42 rest snuggly within the holder bottom 2 and can be removed from the ultra-violet sponge holder 10 and contains a sponge holder drainage opening 44, in which liquid is drained from sponge and held in the drainage retention area 26. FIG. 12 is an exploded view of ultra-violet sponge holder 10 with removable sponge holder 42 exposing the drainage retention area 26 and battery container 22. FIG. 13 is a cross sectional view of ultra-violet sponge holder 10 with removable sponge holder 42 holding sponge 14 and exposing battery 24.

What is claimed is:

1) A ultra-violet sponge holder comprising:
a) a ultra-violet light;
b) a power source;
c) a holder top;
d) a holder bottom;
e) a drainage retention area;
f) a sponge holder:

2) The ultra-violet sponge holder of claim 1, wherein said sponge holder is contained within said holder bottom;

3) The ultra-violet sponge holder of claim 2, wherein said ultra-violet light is contained within said holder top;

4) The ultra-violet sponge holder of claim 3, wherein said drainage retention area is contained within said holder bottom;

5) The ultra-violet sponge holder of claim 4, wherein said power source is a battery which is rechargeable having a rechargeable site;

6) The ultra-violet sponge holder of claim 5, wherein said holder bottom contains a on/off/auto switch;

7) The ultra-violet sponge holder of claim 6, wherein said sponge holder is a removable sponge holder;

8) The ultra-violet sponge holder of claim 7, wherein said removable sponge holder contains a sponge holder drainage opening,

9) The ultra-violet sponge holder of claim 8, wherein said holder bottom contains a holder bottom drainage;

10) The ultra-violet sponge holder of claim 9, wherein said holder top contains a holder top window, whereby allowing the ultra-violet sponge holder to be mobile;

11) The ultra-violet sponge holder of claim 10, wherein said holder top has a reflective top coating and said holder bottom has a reflective bottom coating;

12) The ultra-violet sponge holder of claim 11, wherein said holder bottom contains a bottom ultra-violet light;

13) A ultra-violet sponge holder consists of a holder top and a holder bottom with a ultra-violet light located on said holder top, wherein said holder bottom and said holder top are hinged together to allow to open and close, with a reflective top coating on said holder top and a reflective bottom coating on said holder bottom for increased sterilization of sponge surface, wherein said holder top contains said ultra-violet light powered by a battery through rechargeable means having a rechargeable site to activate said ultra-violet light and said holder bottom contains a sponge holder where a sponge is placed to be sterilized through a on/off/auto switch, which activates the ultra-violet light and a drainage retention area located within said holder bottom to hold drainage from sponge;

14) The ultra-violet sponge holder of claim 13, wherein said holder top contains a holder top window, which can be opened and closed to allow for the sterilization of exterior surfaces, whereby allowing the ultra-violet sponge holder to be mobile;

15) The ultra-violet sponge holder of claim 14, wherein said sponge holder is a removable sponge holder, which can be removed from said ultra-violet sponge holder and contains a sponge holder drainage opening to allow for liquid to be drained from sponge;

16) The ultra-violet sponge holder of claim 15, wherein said holder bottom contains a holder bottom drainage, whereby liquids collected from in said drainage retention area can be drained away from ultra-violet sponge holder,

17) The ultra-violet sponge holder of claim 16, has plurality of said ultra-violet light.

18) A ultra-violet sponge holder having the ability to sterilize the internal environment within the ultra-violet sponge holder and external environment comprising of a ultra-violet light located on a holder top wherein said holder top and a holder bottom are hinged together to allow to open and close, with a reflective top coating on said holder top and a reflective bottom coating on said holder bottom for increased sterilization effect, wherein said holder top contains said ultra-violet light which is powered by a battery located in a battery container through rechargeable means having a rechargeable site to activate said ultra-violet light and said holder bottom contains a on/off/auto switch, which activates said ultra-violet light wherein said holder bottom contains a removable sponge holder containing a sponge holder drainage opening to allow the liquid from said sponge to drain through to a drainage retention area located at the bottom of said holder bottom and said holder top containing a holder top window, which can be opened and closed to sterilize exterior surfaces, whereby allowing the ultra-violet sponge holder to be mobile;

19) The ultra-violet sponge holder of claim 18, wherein said holder bottom also contains a holder bottom drainage, whereby the liquid drams away from said ultra-violet sponge holder;

20) The ultra-violet sponge holder of claim 19, having plurality of said ultra-violet light.
